(19) United States
(12) Patent Application Publication
(10) Pub. No.: US 2014/0100656 A1
(43) Pub. Date: Apr. 10, 2014

(54) RESTORATIVE POST-LUMPECTOMY IMPLANT DEVICE

(71) Applicant: Innovative Biologics LLC, Atlanta, GA (US)

(72) Inventors: James D. Namnoum, Atlanta, GA (US); William A. Barber, Atlanta, GA (US)

(73) Assignee: Innovative Biologics LLC, Atlanta, GA (US)

(21) Appl. No.: 14/039,246

(22) Filed: Sep. 27, 2013

Related U.S. Application Data
(60) Provisional application No. 61/709,603, filed on Oct. 4, 2012.
(60) Provisional application No. 61/716,658, filed on Oct. 22, 2012.

(51) Int. Cl. A61F 2/12

(52) U.S. Cl. CPC A61F 2/12
USPC 623/8

(57) ABSTRACT

Provided is a restorative breast implant device that can be used to replace lumpectomy tissue and prevent the late aesthetic deformities which may occur following lumpectomy or partial mastectomy. The disclosed implant is an inflatable device comprising an outer shell composed of a biological material and an inner chamber. The device may be inflated/filled with a biological filler material to conform the implant to a lumpectomy cavity's dimensions. In addition, the disclosed implant is able to attain a blood supply thereby insuring incorporation into the breast while resisting resorption. The restorative breast implant is also optionally radiolucent so as not to interfere with future surveillance imaging. Further, in contrast to synthetic radiopaque implants, the disclosed implant resists fibrosis and infection.

### CROSS-REFERENCE TO RELATED APPLICATIONS

This application claims benefit of U.S. Provisional Application No. 61/709,603, filed Oct. 4, 2012, and U.S. Provisional Application Ser. No. 61/716,658, filed Oct. 22, 2012, which are hereby incorporated herein by reference in their entirety.

### FIELD

The present application relates generally to devices, systems, and methods for using a restorative breast implant to replace breast lumpectomy (partial mastectomy) tissue.

### BACKGROUND

Lumpectomy of the breast for cancer is a surgical treatment technique to remove the portion of the breast affected by cancer with a zone of surrounding normal tissue thereby rendering it cancer free while preserving the reminder of the breast uninvolved with disease. When coupled with whole breast radiation therapy or radiation delivered locally, the survival rates for appropriately selected patients are equivalent to mastectomy. Selection of either lumpectomy or mastectomy for treatment of breast cancer is based on a number of variables. A lumpectomy to remove a tumor can drastically disfigure the breast. The potential breast deformity (size discrepancy) following lumpectomy is one of the principal determinants affecting the selection process.

The volume of tissue removed during a lumpectomy ranges in size from about 2 cc to about 400 cc, more commonly from the size of a walnut (33 cc) to the size of a tangerine (85 cc), and leaves behind a cavity corresponding to that volume plus an additional deficit due to collateral atrophy from cautery use. Initially, the defect created fills with fluid in response to the injury. Over time, however, the fluid is reabsorbed resulting in a cavity that collapses due to a lack of structural support. This collapse is manifested topographically by distortion of the remaining breast architecture leading to a number of problems such as nipple deformity, breast deformity, and asymmetry with the opposite breast. This asymmetry can also cause problems with the proper fit of garments.

The location of tissue removal and the pre-existing breast size are significant determinants of the aesthetic deformity that ensues. In most cases, radiation either through a whole breast (external beam) or a partial breast (local irradiation) approach further compounds the acquired deformity by inducing shrinkage, fibrosis, and contraction of the breast and lumpectomy cavity. Little can be done effectively to restore the normal breast contour once this process is completed. The resulting deformity can be considered permanent.

Treatment options to correct the deformity can be performed immediately after the removal of the lumpectomy specimen in some cases (oncoplastic approach) if the following conditions are met: the breast is large enough; the surgeon has adequate training or a plastic surgeon is consulted with training in this area; the patient is willing to have additional breast scars and internal breast scarring from tissue rearrangement that may reduce effectiveness of future mammographic surveillance; and the patient is willing to undergo a balancing procedure on the opposite breasts with attendant scars and risk. Procedurally, the oncoplastic approach is impractical in most cases for these reasons and the general lack of training among breast oncologic surgeons as well as the difficulties of coordinating surgery with the plastic surgeon.

### SUMMARY

Provided is a restorative breast implant device that can be used to replace breast lumpectomy tissue that prevents the late aesthetic deformities which may occur following lumpectomy or partial mastectomy. Use of the device can greatly improve the aesthetics of the breast and for that reason increase the number of candidates for breast conserving surgery.

The disclosed implant is an inflatable device (e.g., a balloon, pillow, or bag) comprising an outer shell composed of a biological material and an inner chamber. The device may be inflated/filled with a filler material to conform the implant to a lumpectomy cavity's dimensions. In addition, the disclosed implant may be vascularized thereby insuring incorporation into the breast while resisting resorption. The restorative breast implant is also optionally radiolucent so as not to interfere with future surveillance imaging. Further, in contrast to synthetic implants, the disclosed implant resists fibrosis and infection.

The disclosed implant is optionally inflated/filled in situ after implantation. Therefore, the disclosed implant may be implanted percutanously. However, a pre-filled implant may also be implanted intraoperatively.

The disclosed implant can be placed either immediately after the removal of the tissue or at some point post-operatively, e.g., after the removal of a partial breast radiotherapy balloon. Immediate to near immediate volume restoration can prevent subsequent collapse of the lumpectomy cavity following cavity fluid reabsorption and radiation induced fibrosis and shrinkage.

These and other features and advantages of embodiments of the present disclosure will become more readily apparent to those skilled in the art after consideration of the following detailed description and accompanying drawings, which describe both the preferred and alternative implementations of the present disclosure.

### BRIEF DESCRIPTION OF THE DRAWINGS

FIGS. 1A and 1B are perspective views of exemplary restorative post-lumpectomy implant devices attached to a tube (FIG. 1A) or catheter (FIG. 2B) and a syringe for in situ inflation.

FIGS. 2A, 2B, 2C, and 2D are perspective (FIG. 2A), cross-sectional (FIG. 2B and 2C), and partially cut-away (FIG. 2D) views of an exemplary restorative post-lumpectomy implant device.

FIG. 3 is a diagram illustrating an exemplary procedure for implanting a restorative post-lumpectomy implant device into the void created by a lumpectomy and optional regional radiotherapy followed by in situ inflation.

FIG. 4 is a cross-section view of an exemplary percutaneous insertion device for implanting a restorative post-lumpectomy implant device

### DETAILED DESCRIPTION

The present disclosure now will be described more fully hereinafter. Indeed, these implementations can be embodied in many different forms and should not be construed as limited to the implementations set forth herein; rather, these implementations are provided so that this disclosure will satisfy applicable legal requirements. As used in the specification, and in the appended claims, the singular forms “a”, “an”, “the”, include plural referents unless the context clearly dictates otherwise. The term “comprising” and variations thereof as used herein is used synonymously with the term “including” and variations thereof and are open, non-limiting terms.

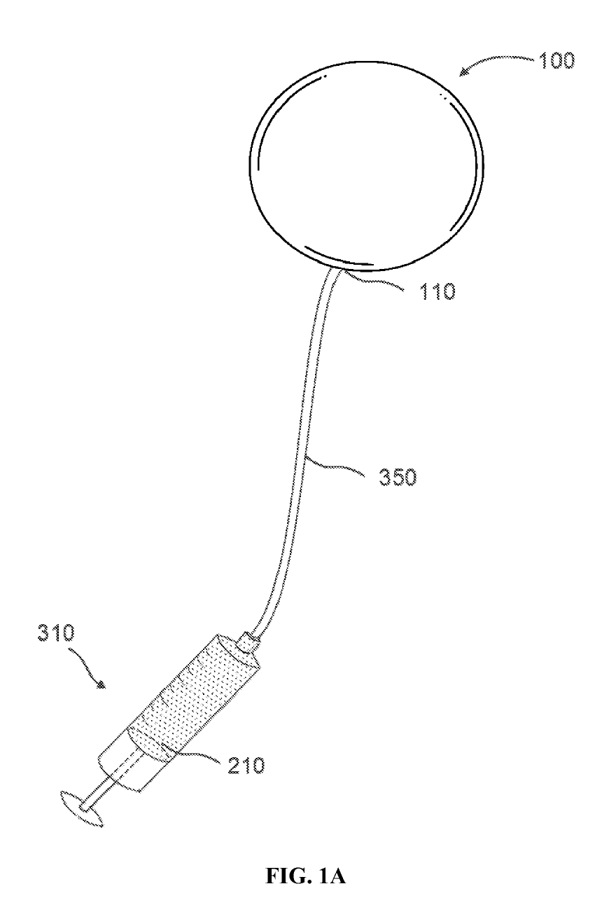

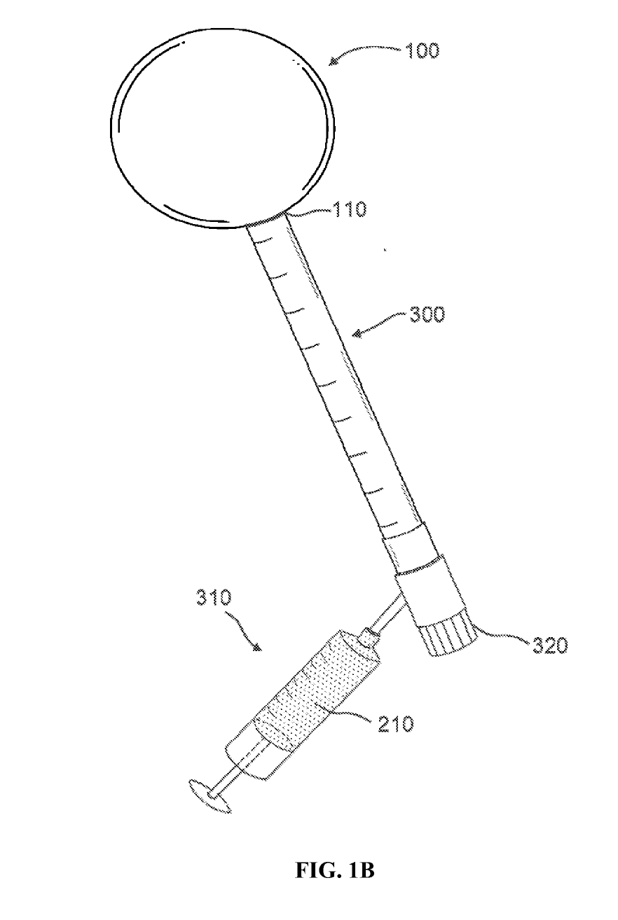

FIGS. 1A and 1B are perspective views of two exemplary embodiments of a restorative breast implant 100 fluidly attached by a valve 110 to either tubing 350 (FIG. 1A) or a catheter 300 (FIG. 1B) for use as an implant to fill a void created by a lumpectomy or partial mastectomy. Also shown in FIGS. 1A and 1B is a syringe 310 containing filler material 210 fluidly attached to the tubing 350 (FIG. 1A) or catheter 300 with a guide rod 320 engaged within the catheter 300 (FIG. 1B). These embodiments allow for in situ inflation of the restorative breast implant 100 after optional percutaneous delivery.

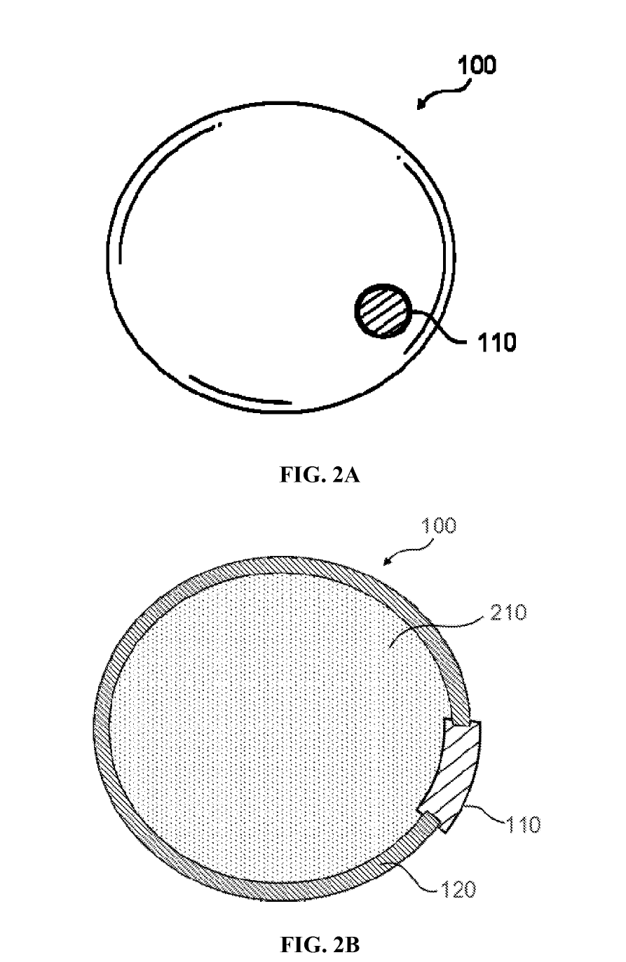

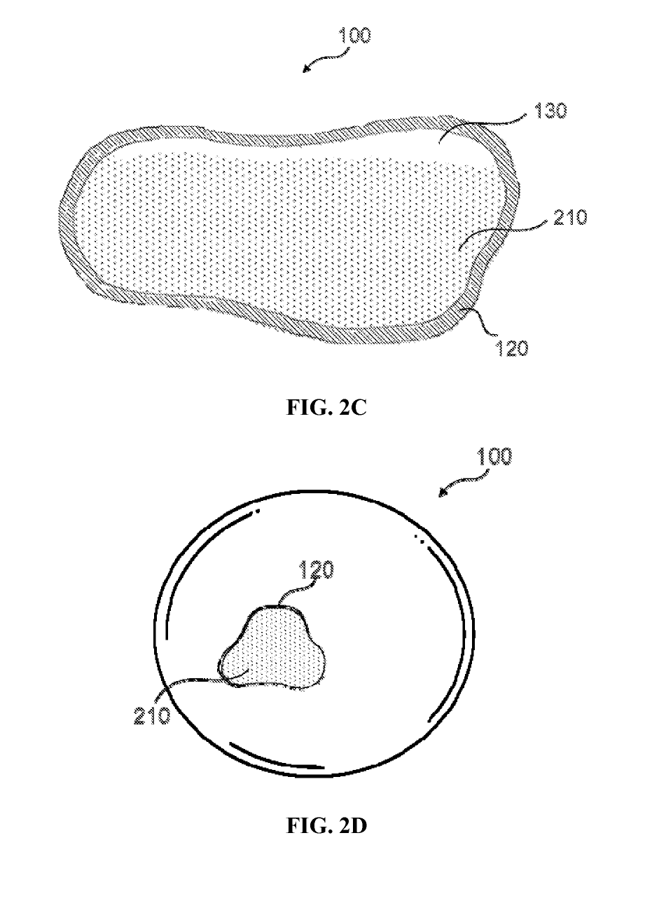

FIG. 2A is a perspective view of an exemplary restorative breast implant 100 containing a valve 110. As shown in FIGS. 2B-2C, the restorative breast implant 100 is an inflatable balloon/pillow composed of an outer shell wall 120 and an inner chamber 130 that can be inflated/filled with a filler material 210. FIGS. 2B and 2C are cross-sectional views of the restorative breast implant 100 when fully inflated/filled (FIG. 2B) or partially inflated/filled (FIG. 2C) with a filler material 210. Also shown in FIG. 2B is a cross-sectional view of an exemplary valve 110. FIG. 2D is a partially cut-away view of the restorative breast implant 100 showing the outer shell wall 120 and filler material 210 within the inner chamber 130.

Referring again to FIG. 2B, when fully inflated/filled, the restorative breast implant 100 can have a generally spherical shape with a volume of about 5 cm³ to about 400 cm³ (5 ml to 400 ml), including about 30 cm³ to 400 cm³ (30 ml to 400 ml), and 33 cm³ to about 85 cm³ (33 ml to 85 ml). Therefore, the restorative breast implant 100 can have a maximum volume of 30 ml to 400 ml, including 33 to 85 ml). However, other shapes and volumes are contemplated in order to conform the restorative breast implant 100 to a lumpectomy cavity's dimensions. The disclosed restorative breast implant 100 may be partially or fully inflated/filled with the filler material 210.

The outer shell wall 120 is composed of a pliable, optionally elastic, biomaterial that is non-immunoreactive and promotes vascularization. The outer shell wall 120 biomaterial can be a biological scaffold obtained from mammals or insects. Examples of biological scaffolds that can be obtained from mammals include decellularized dermis, mesothelium, or submucosa (e.g., urinary bladder, intestinal, or stomach). For example, the biomaterial may be acellular human dermis, such as ALLODERM Tissue Matrix (Life Cell, Branchburg, N.J.), MEDOR Matrix (Kensey Nash, Exton, Pa.), or DERMAMATRIX Acellular Dermis (Musculoskeletal Transplant Foundation®, DePuy SYnthes, West Chester, Pa.). The biomaterial may be mesothelium extracellular matrix, such as MESO BIOMATRIX (Kensey Nash, Exton, Pa.)). Alternatively, the biomaterial may be acellular non-human dermis, e.g., from a bovine or porcine animal, such as SURGIMEND, PRIMATRIX, DUREPAIR, XENFORM, or TISSUEMEND (TEI Biosciences, Boston, Mass.). A suitable biomaterial (e.g., mesh) may also be produced from a bioengineered silk, such as SERISCAFFOLD (Allergan Medical, Irvine Calif.).

The filler material 210 is an injectable liquid or semi-solid biological material that is non-immunoreactive and promotes vascularization. For example, the filler material 210 may be a collagen, hyaluronic acid gel, lyophilized dermis, biological polymer, stroma, collagenous soft tissue lattice, adiopose tissue, silk, or a combination thereof. The filler material may be composed of particles (e.g., beads or granules) that arrange themselves in an ordering fashion to fill a space with the correct volume. The particles may be, for example, nanoparticles, microparticles, or combinations thereof. Therefore, in some embodiments, the particles have a mean diameter of about 1 nm to about 1 cm, including about 10 nm to about 10 μm, or about 100 nm to about 1 μm. The particles may be suspended in an injectable liquid or semi-solid material, such as a gel. The particles may be spherical or non-spherical.

The filler material may contain cells, such as stem cells, progenitor cells, fat cells, or a combination thereof. The outer shell wall 120 biomaterial, the filler material 210, or a combination thereof, optionally contains growth factors that promote angiogenesis and vascularization of the implant 100. In some cases, the implant 100 releases vascular endothelial growth factor (VEGF). Therefore, in some cases, the filler material 210 contains cells containing recombinant expression vectors encoding one or more growth factors promote angiogenesis, such as VEGF.

The outer shell wall 120 biomaterial and the filler material 210 can be derived from a homologous, autologous, or heterologous sources. In some cases, the outer shell wall 120 biomaterial and/or the filler material 210 is a xenograft derived from a non-human mammal, such as a bovine or porcine animal. However, the outer shell wall 120 biomaterial and/or the filler material 210 may also be an allograft derived from a human source or an autograft derived from the patient's own body.

The outer shell wall 120 biomaterial and the filler material 210 are also optionally radiolucent so as not to interfere with future surveillance imaging. For example, the restorative breast implant 100 optionally has a radiodensity less than a silicone breast implant.

The restorative breast implant 100 may be pre-filled at standard volumes for implantation without in situ inflation. This approach requires a larger incision for implantation, but avoids the need for a valve 110. Therefore, restorative breast implants 100 are disclosed that lack a valve 110 and instead contain a fixed volume of filler material 210.

The valve 110, when used, is a one-way or two-way valve that allows the physician to fill, and optionally empty, the restorative breast implant 100 with filler material 210. For example, the valve 110 can be a leaf valve, a kink valve, or a diaphragm valve. The valve may be produced from a biocompatible synthetic material, such as silicone. Optionally, the valve is made from a biological material, such as those used to form the outer shell wall 120.

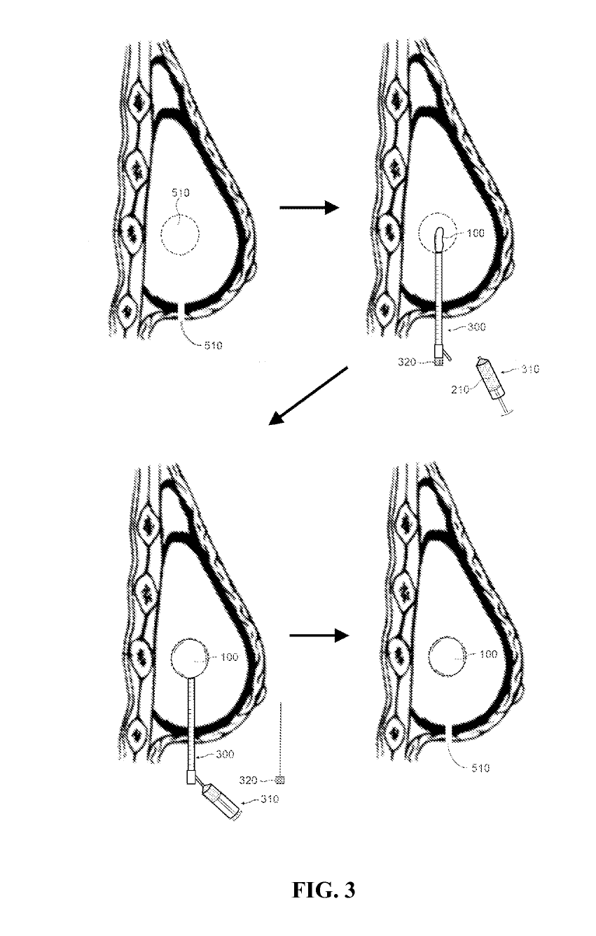

According to some embodiments, the restorative breast implant 100 is inflated/filled in situ with the filler material 210 after implantation within a cavity 500 created by a lumpectomy or partial mastectomy. FIG. 3 is a diagram illustrating an exemplary procedure for implanting a restorative post-lumpectomy implant device. A deflated (unfilled or partially filled) restorative breast implant 100 is inserted through an incision 510 in the breast and guided to the lumpectomy cavity 500 using a catheter 300 and optional guide rod 320 attached to the restorative breast implant 100 by a valve 110 within the implant. The guide rod 320, if used, is then retracted from within the catheter and discarded. A syringe is then attached to the catheter 300 and used to inflate the restorative breast implant 100 with a sufficient amount of filler material 210 to fill the lumpectomy cavity 500. In other embodiments, a catheter and guide rod are not needed, so the deflated (unfilled or partially filled) restorative breast implant 100 is fluidly attached to the syringe 310 by tubing 350 connected to the valve 110.

A suitable volume of filler material 210 can be determined by visual inspection of the lump removed, the dimensions of the cavity being filled, by viewing the contour of the breast after correction with the implant, or any combination thereof. Once inflated, the catheter is detached from the valve 110 and removed from the breast. Optionally, the fill volume is subsequently adjusted to correct deformity. For example, a two-way valve may be used to permit adjustments up and down in volume as needed so long as the syringe is fluidly connected to the valve 110 on the implant. After the implant is placed and the volume optimized, the syringe 310 and catheter 300 is disconnected from the valve thereby sealing the valve and implant. The skin may then be closed over the implant, e.g., in two layers using interrupted and running inter-dermal sutures or two layers of running inter-dermal sutures. Wound sealant may also be placed, and antibiotics may be given prophylactically.

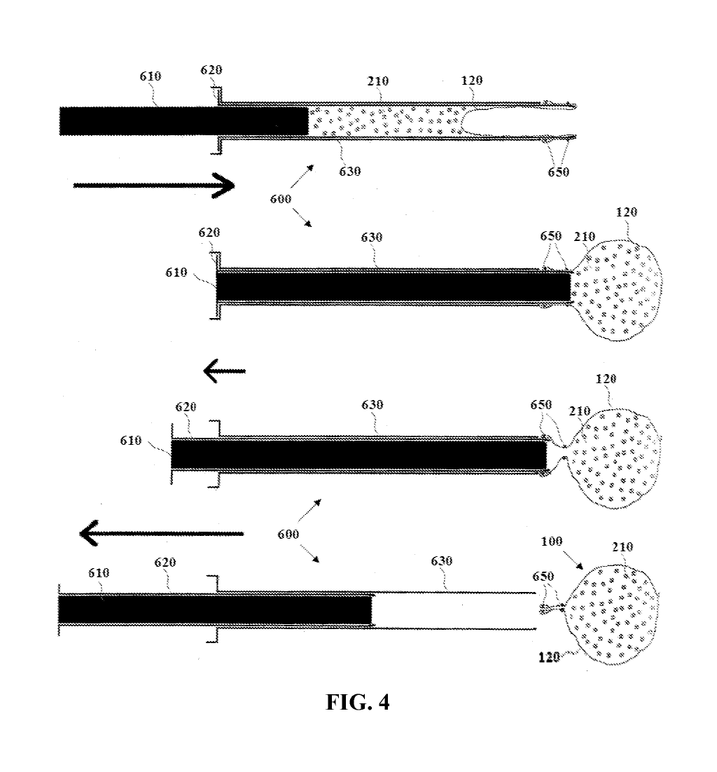

FIG. 4 is a cross-section view of an exemplary percutaneous insertion device 600 for implanting a restorative breast implant 100. The percutaneous insertion device 600 can be used to inflate/fill outer shell wall 120 biomaterial with the filler material 210 after percutaneous insertion. According to some embodiments, the percutaneous insertion device 600 comprises a dual-lumen tube having inner tube 620 and an outer tube 630, wherein the inner tube 620 is sized and configured to slidably pass through the lumen of the outer tube 630. The percutaneous insertion device 600 can also comprise a plunger 610 sized and configured to slidably pass through the lumen of the inner tube 620 so as to force filler material 210 through the inner tube 620 out its distal end. In preferred embodiments, the distal end of the inner tube 620 can extend beyond the distal end of the outer lumen 630 when the inner tube 620 is fully interposed within the outer tube 630. In this conformation, the outer shell wall 120 can be secured to the outer surface of the inner tube 620 at its distal end by one or more elastics 650. When the inner tube 620 is retracted in the proximal direction, the distal end of the outer tube 630 advances over the distal end of the inner tube 620 and displaces the one or more elastics 650 from the outer surface of the inner tube 620. The percutaneous insertion device 600 can therefore be used to inflate/fill outer shell wall 120 biomaterial with the filler material 210 by first injecting the percutaneous insertion device 600 into a void created by a lumpectomy, advancing the plunger 610 to force filler material 210 through the inner tube 620 out its distal end into the outer shell wall 120 secured to the outer surface of the inner tube 620 at its distal end by one or more elastics 650. Once the breast implant 100 is fully filled/inflated, the inner tube 620 is retracted to displace the one or more elastics 650 from the outer surface of the inner tube 620, which creates a seal in the breast implant 100.

The disclosed restorative breast implant 100 may be implanted within a void created by a lumpectomy or partial mastectomy procedure any time after surgery, but is preferably implanted immediately or nearly immediately after a lumpectomy procedure or partial mastectomy. The term “immediate” as used herein refers to the same day as a medical procedure, i.e., while the patient is still in the operating room or doctor's office. The term “near immediate” includes a time period from 1 day to 2 weeks after a medical procedure.

Also disclosed is a method for immediate to near immediate implant reconstruction following local/regional radiotherapy to the tumor bed using a catheter based implant device, such as MAMMOSITE (Hologic, Bedford, Mass.). For example, following removal of the MAMMOSITE device and cavity washout with antimicrobial solution (e.g., bacitracin/betadine), a deflated or partially inflated restorative breast implant 100 fluidly attached to the end of a deployable catheter 300 via a valve 110 may be inserted through the incision 510 used for the MAMMOSITE (approximately 2.5 cm) and placed into the lumpectomy cavity 500. A deployable guide rod 320, if used, is then retracted from within the catheter and discarded. A syringe 310 may then be attached to the catheter 300 and the filler material 210 injected into the implant 100, filling it to the desired amount for optimal volume correction. Volume adjustments are possible until the fill catheter 300 and syringe 310 are removed. After the implant is placed and the volume optimized, the syringe 310 and catheter 300 are disconnected from the valve 110 thereby sealing the valve 110 and implant 100. The skin may then be closed over the implant 100, e.g., in two layers using interrupted and running inter-dermal sutures or two layers of running inter-dermal sutures. Wound sealant may also be placed, and antibiotics may be given prophylactically.

What is claimed is:

1. A restorative breast implant device comprising:
(a) an outer shell formed from a pliable, non-immunoreactive biological material that promotes vascularization, and
(b) an inner chamber optionally comprising a filler material comprising an injectable liquid or semi-solid biological material.

2. The device of claim 1, further comprising a valve configured for inflation of the implant device with the filler material.

3. The device of claim 1, partially or fully inflated with the filler material.

4. The device of claim 1, wherein the pliable biological material is an acellular biological scaffold.

5. The device of claim 4, wherein the pliable biological material is decellularized dermis, mesothelium, or submucosa.

6. The device of claim 4, wherein the pliable biological material is acellularized human dermis.

7. The device of claim 1, wherein the pliable biological material is a mesh or fabric woven from a biological polymer.

8. The device of claim 7, wherein the biological polymer is a bioengineered silk.

9. The device of claim 1, wherein the filler material is non-immunoreactive and promotes vascularization.

10. The device of claim 9, wherein the filler material is a collagen, a hyaluronic acid gel, or a fat.

11. The device of claim 1, wherein the filler material comprises nanoparticles, microparticles, or a combination thereof, suspended in the injectable liquid or semi-solid biological material.

12. The device of claim 11, wherein the nanoparticles, microparticles, or a combination thereof, comprise silk.

13. The device of claim 1, wherein the inner chamber has a maximum volume of 30 ml to 400 ml.

14. The device of claim 1, further comprising a catheter fluidly connected to the valve.

15. The device of claim 14, further comprising a syringe fluidly connected to the catheter, wherein the syringe comprises the filler material, wherein depression of the syringe inflates the implant device.

16. A method for restoring a subject's breast after a lumpectomy, comprising
(a) implanting within a lumpectomy cavity the implant device of claim 1; and
(b) inflating the implant device with an effective amount of filler material to conform the outer shell to the lumpectomy cavity dimensions.

17. The method of claim 16, wherein the device is implanted within the lumpectomy cavity by inserting through an incision a catheter fluidly connected to the inner chamber of the device.

18. The method of claim 17, wherein the incision was first used to insert a device for regionally irradiating the lumpectomy cavity, wherein the restorative breast implant device is implanted within the lumpectomy cavity after the final radiation dosage and before the incision is sutured.
